(19) United States
(12) Plant Patent Application Publication
(10) Pub. No.: US 2016/0278269 P1
(43) Pub. Date: Sep. 22, 2016

(54) Osteospermum Plant Named 'KLEOE14235'

(50) ;

(71) Applicant: Klemm+Sohn GmbH & Co. KG, Stuttgart (DE)

(72) Inventors: Nils Klemm, Stuttgart (DE); Silke Wagener, Leinfelden-Echterdingen (DE)

(73) Assignee: Klemm+Sohn GmbH & Co. KG, Stuttgart (DE)

(21) Appl. No.: 14/545,027

(22) Filed: Mar. 18, 2015

(51) Int. Cl. A01H 5/00

(52) U.S. Cl. PLT/360

(57) ABSTRACT

A new osteospermum plant particularly distinguished by light violet ray florets with a lighter violet ring and darker at the base, and a semi-upright growth habit, is disclosed.

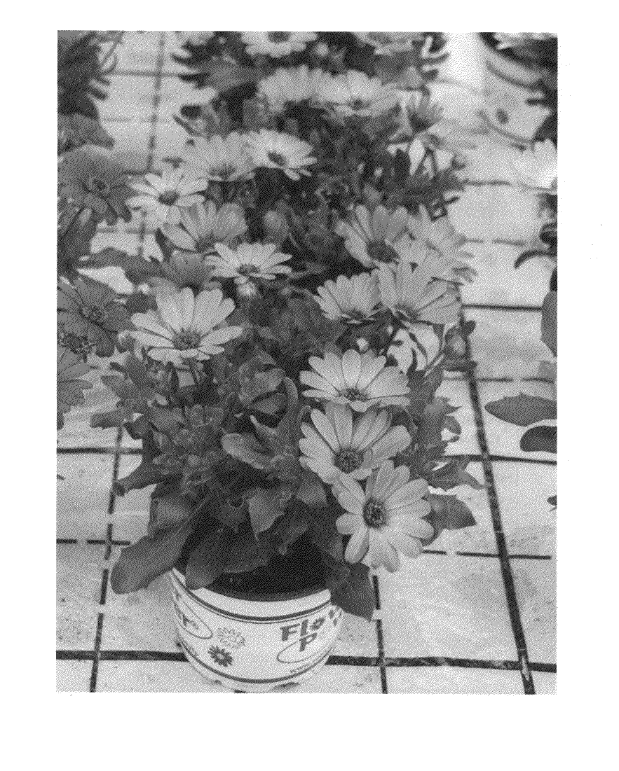

### GENUS AND SPECIES

Osteospermum ecklonis Norl

### VARIETY DENOMINATION

‘KLEOE14235’

### BACKGROUND OF THE NEW PLANT

The present invention comprises a new and distinct variety of osteospermum, botanically known as Osteospermum ecklonis Norl, and hereinafter referred to by the variety name ‘KLEOE14235’. ‘KLEOE14235’ originated from a cross-pollination conducted in the summer of 2009 in Stuttgart, Germany between the proprietary female osteospermum variety ‘0E-2006-0062’ (unpatented), and the proprietary male osteospermum variety ‘OE-2001-U 088’ (unpatented).

The seeds from the cross were sown and plants were grown in a greenhouse for evaluation, where an individual plant designated ‘KLEOE14235’ was selected from the group of plants in April 2011 in Stuttgart, Germany. In May 2011 in Stuttgart, Germany, ‘KLEOE14235’ was first vegetatively propagated by terminal cuttings. ‘KLEOE14235’ was found to reproduce true to type in successive generations of asexual propagation via terminal cuttings.

### SUMMARY

The following are the most outstanding and distinguishing characteristics of this new variety when grown under normal horticultural practices in Stuttgart, Germany.

1. Light violet ray florets with a lighter violet ring and darker at the base; and 2. A semi-upright growth habit.

### DESCRIPTION OF THE PHOTOGRAPH

This new osteospermum plant is illustrated by the accompanying photograph which shows the plant's overall plant habit including form, foliage, and flowers. The photograph is of a three-month-old plant grown in a greenhouse in Stuttgart, Germany in June 2012. The colors shown are as true as can be reasonably obtained by conventional photographic procedures.

### DESCRIPTION OF THE NEW VARIETY

The following detailed descriptions set forth the distinctive characteristics of ‘KLEOE14235’. The data which define these characteristics were collected from asexual reproductions carried out in Stuttgart, Germany. Data was collected on 5-month-old plants grown in a greenhouse using a growth regulation of tilt 0.5% and 0.8% several times in Stuttgart, Germany in March 2015. Color references are to The R.H.S. Colour Chart of The Royal Horticultural Society of London (R.H.S.), 5^{th} edition (2007).
Classification:
   Family.—Asteraceae.
   Botanical.—Osteospermum ecklonis Norl.
   Common.—African Daisy.
   Designation.—‘KLEOE14235’.
Parentage:
   Female parent.—The proprietary osteospermum variety ‘0E-2006-0062’ (unpatented).
   Male parent.—The proprietary osteospermum variety ‘0E-2001-U 088’ (unpatented).
Plant:
   Form and habit.—Herbaceous perennial but usually cultivated as an annual; not frost resistant.
   Growth habit.—Semi-upright.
   Branching habit.—Freely branching.
   Height (from top of soil, including inflorescence).—About 30.0 cm.
   Width (including inflorescences).—About 25.0 cm.
   Propagation.—Terminal cuttings.
   Time to produce a finished flowering plant.—14 weeks (in the spring and winter).
   Time to initiate and develop roots.—3 weeks.
   Root description.—Freely rooting, light tan in color.
Leaves:
   Arrangement.—Alternate.
   Shape.—Obovate.
   Apex.—Broadly acute.
   Base.—Narrowly acute.
   Margin.—Shallow.
   Immature color.—Upper surface: RHS 143A Lower surface: RHS 143B.
   Mature color.—Upper surface: RHS 141A Lower surface: RHS 139B.
   Length.—Up to 7.0 cm.
   Width.—Up to 3.0 cm.
   Texture and appearance.—Upper surface: Shiny leathery Lower surface: Pubescent; covering of fine hairs.
   Petioles.—Absent.
   Venation pattern.—Pinnate.
   Venation color (both upper and lower surfaces).—RHS 143C.
Stems:
   Total number of branches per plant (calculated at the base of the plant).—5.
   Length.—Up to 20.0 cm.
   Diameter.—0.6 cm.
   Internode length.—About 1.5 cm.
   Color.—RHS 143C.
   Texture.—Shiny, smooth.
   Anthocyanin.—Absent.
Flower buds:
   Shape.—Round-ovoid.
   Length.—About 1.0 cm.
   Diameter.—About 1.0 cm.
   Color (at tight bud just before the ray florets unfold).—RHS 143A.
Inflorescence:
   Type.—Composite, single.
   Blooming habit.—Spring to autumn.
   Quantity of inflorescences per plant.—30 to 40.
   Lastingness of the inflorescences on the plant.—About 2 weeks.
   Fragrance.—Absent to faint.
   Inflorescence diameter.—About 6.0 cm.
   Inflorescence depth.—1.0 cm to 1.5 cm.
   Disc diameter.—About 1.7 cm.
Disc floret:
   Quantity per inflorescence.—About 110.
   Shape.—Oblanceolate.
   Tube color.—Closed: RHS 139D Mature: RHS 93B.
   Length.—About 0.5 cm.
   Diameter (at apex).—About 0.1 cm.
   Apex.—Obtuse.
   Apex color.—RHS 93B.
   Base.—Acute.
   Base color.—RHS 155C.
   Margin.—Entire.
Ray floret:
   Quantity per inflorescence.—About 22.
   Shape.—Oblanceolate.
   Color.—Upper surface: Apical and middle part RHS 84B, basal RHS 84C, darker ring at base RHS 80B, and end of flowering RHS N78B. Lower surface: RHS N82A.
   Length.—About 2.5 cm.
   Width.—1.8 cm.
   Apex.—Obtuse.
   Base.—Acute.
   Margin.—Entire.
   Texture.—Upper surface: Smooth Lower surface: Smooth, shiny.
Peduncle:
   Length.—Up to 8.0 cm.
   Diameter.—About 0.2 cm.
   Texture.—Pubescent; covering of fine hairs.
   Color.—RHS 143C.
Phyllaries:
   Texture.—Pubescent; covering of fine hairs.
   Arrangement.—Single.
   Observed quantity per flower.—About 20.
   Shape.—Lanceolate.
   Color.—Outer surface: RHS 143A Inner surface: RHS 143B.
   Length.—Between 0.7 cm and 1.2 cm.
   Width.—Up to 0.4 cm.
   Apex.—Narrowly acute.
   Base.—Acute.
   Margin.—Entire.
   Texture.—Outer surface: Pubescent; covering of fine hairs Inner surface: Shiny.
Reproductive organs:
   Androecium.—Location: On the disc floret Stamens: Quantity: Numerous Shape: Filamentous Filament length: About 0.4 cm Filament diameter: Less than 0.05 cm Anther: Shape: Lanceolate Color: RHS N92A Length: About 0.2 cm Diameter: Less than 0.05 cm Pollen: Color: RHS 14A Amount: Moderate.
   Gynoecium.—Location: On the base of the petals (ray floret), reduced on disc florets Pistils: Number: About 22 per flower, one per petal Length: About 0.5 cm Diameter: Less than 0.05 cm Stigma: Color: RHS N92A Shape: Split in two parts at the end Length: About 0.2 cm Diameter: Less than 0.05 cm Style: Color: RHS 76B Length: About 0.3 cm Diameter: 0.05 cm.
Fruit and seed set: None observed.
Disease and pest/insect resistance: None observed.

### COMPARISON WITH PARENTAL LINES AND KNOWN VARIETY

‘KLEOE14235’ is a distinct variety of osteospermum and is distinguished from its parents as described in Table 1.

TABLE 1
Comparison with Parental Lines

| | | Female Parent | Male Parent |
|---|---|---|---|
| Characteristic | ‘KLEOE14235’ | ‘OE-2006-0062’ | ‘OE-2001-U 088’ |
| Ray floret | Light violet with | Pink-violet | Pink with very |
| color | lighter violet ring | | broad white ring |
| | basal and darker | | |
| | at base | | |

‘KLEOE14235’ is most similar to the commercial osteospermum variety ‘KLEOE05115’ (U.S. Plant Pat. No. 18,634). Differences between the varieties are described in Table 2.

TABLE 2
Comparison with Similar Variety

| Characteristic | ‘KLEOE14235’ | ‘KLEOE05115’ |
|---|---|---|
| Plant height | Short to medium; about | Medium to tall; about |
| | 30.0 cm tall | 40.0 cm tall |

We claim:

1. A new and distinct variety of osteospermum plant designated ‘KLEOE14235’ as illustrated and described herein.
